(19) United States
(12) Patent Application Publication
(10) Pub. No.: US 2017/0350908 A1
(43) Pub. Date: Dec. 7, 2017

(54) QUANTIFYING PROTEIN ISOFORMS

(71) Applicant: Optys Tech Corporation, Brighton, MA (US)

(72) Inventor: Amol Prakash, Brighton, MA (US)

(21) Appl. No.: 15/613,447

(22) Filed: Jun. 5, 2017

Related U.S. Application Data
(60) Provisional application No. 62/345,444, filed on Jun. 3, 2016.

(51) Int. Cl. G01N 33/92; G01N 33/68; G06F 19/22

(52) U.S. Cl. CPC G01N 33/92; G06F 19/22; G01N 33/6842; G01N 2333/775; G01N 2570/00

(57) ABSTRACT

Provided herein are methods for determining the ratio of one or more isoforms of a protein in a sample.

### RELATED APPLICATIONS

This application claims priority to U.S. Provisional Patent Application No. 62/345,444, filed Jun. 3, 2016, the contents of which are incorporated herein by reference.

### BACKGROUND

Quantitative measurement of proteins is an important aspect in the discovery of biomarkers. Mammalian genes can undergo multiple mutations, rearrangements and splicing events, thereby translating into protein isoforms. Knowing the relative or absolute amounts of these protein isoforms can advance biotechnology and clinical domains.

Previous studies to determine protein isoforms include the use of peptides which are unique to each protein isoform as a way to measure each isoform. FIG. 1 is an example of this approach where the entire box represents the protein sequence and the sub-boxes represent different peptide sequences. Some peptides may be common across all or few forms (shown in FIG. 1 by the dark grey box), and some may be unique to one or more form (displayed in FIG. 1 by the light grey box). The problem with this approach, and others, is that not many protein isoforms contain a peptide that unique for each and every isoform. Furthermore, the particular peptide(s) that are unique to an isoform may be below the level of detection or below the quantification limit. Also, developing synthetic standards for every unique peptide is not only costly and time consuming, but can also become impractical for a large number of proteins.

Other methods, such as the use of gel bands, have also been employed to quantify protein isoforms. However, these methods require that the protein isoform is at least separable, or capable of being resolved. Additionally, these methods further require the presence of a common peptide across all of the isoforms. Not to mention that in each of the above methods, as well as other viable methods, isotopic labelling has become the standard for retroanalysis cross-reference to the original isoform.

Because of the drawbacks associated with prior approaches, only limited solutions exist for quantifying protein isoforms. The need therefore remains for more robust and practical methods to determine the presence of, and to quantify, protein isoforms.

### SUMMARY

Provided herein are methods for quantifying protein isoforms using, in part, peptides that belong to one or more isoform and share sequence homology.

This approach provides multiple advantages. For example, the present methods can be used to isolate and quantify protein isoforms from peptides which are not unique to isoform of interest. In other words, the quantitative response from peptides which are not unique to the isoform of interest can be used as a surrogate to identify and quantitate that isoform. In addition, the methods described herein do not require the use of isotopic labels. Therefore, the handling of expensive radioactive materials is minimized. Furthermore, this method does not require peptides which are unique to individual isoforms. Taken together, the methods described herein provide at a minimum, a high-throughput quantification technique which is accurate, cost-effective, and versatile across a variety of samples, including those which consist of highly complex mixtures.

### BRIEF DESCRIPTION OF THE FIGURES

FIG. 1 illustrates protein isoforms containing common and unique properties.

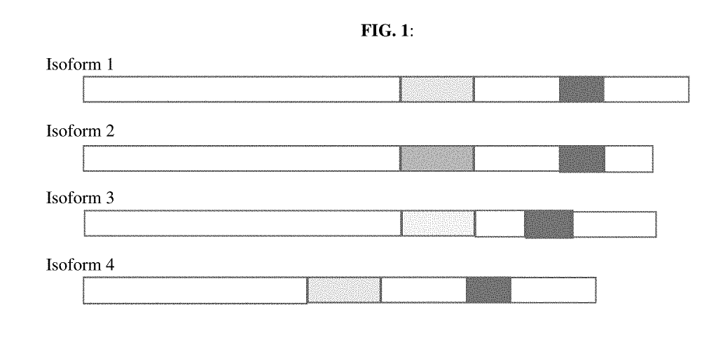

FIG. 2 illustrates an exemplary method for determining the ratio of 4 isoforms of a protein.

### DETAILED DESCRIPTION OF CERTAIN EMBODIMENTS

In a first embodiment, provided herein is a method of determining the ratio of one or more isoforms of a protein in a sample comprising digesting the protein with at least one protease to produce a plurality of peptides; quantifying one of more of the peptides produced; selecting, from the peptides produced, groups of peptides having sequence homology and at least two peptides; identifying, for each selected group of peptides, which peptides belong to which one or more target isoforms; determining the quantitative ratio between peptides belonging to a group; and calculating the ratio of protein isoforms or ratio of groups of protein isoforms, thereby determining the relative ratio of the one or more isoforms.

“Protein isoforms” or “isoforms of a protein” mean proteins that exist or occur in multiple forms. The creation of protein isoforms may occur from e.g., mutations, gene rearrangements, translation from different genes, nucleotide insertion, nucleotide deletion, nucleotide duplication, nucleotide rearrangements, or various splicing events. Isoforms can be naturally observed variants or synthetically created variants.

As used herein, groups of peptides having sequence homology means groups of peptides which only differ in their sequence by a small number of amino acids (including both the location and/or order). Differences can include e.g., amino acid substitutions, deletions, additions or rearrangements. In one aspect, having sequence homology means that the amino acid residues present within a group of peptides are at least 60% the same, including both location and order of the amino acid residues. For example, the amino acid residues present may be at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% the same including both the location and order of the amino acid residues.

It will be appreciated that the higher the percentage of sequence homology, i.e., the lower the amount of differing amino acids between two or more peptides, the more similar the physiochemical properties will be between those peptides. In these instances, similar quantitative responses will be exhibited and can be measured by methods know in the art, e.g., by mass spectrometry. Physiochemical properties include e.g., basicity, hydrophobicity, ionization potential, molecular weight, side chain structure, and the like.

In a second embodiment, at least one peptide group selected from those in the first embodiment having sequence homology and at least two peptides comprises one or more peptides that are unique to particular isoform.

In a third embodiment, the quantitative ratio of two peptides from a single group in the first or second embodiment, is used to calculate and report the ratio of two protein isoforms. Alternatively, the quantitative ratio of two peptides from a single group in the first or second embodiment is used to calculate and report the ratio of two groups of protein isoforms.

In a fourth embodiment, at least one peptide in the selected group of the first embodiment is below the level of detection of quantification. Other features optionally incorporated to this embodiment are as described herein e.g., as in the second or third embodiment. Alternatively, at least one peptide in the selected group of the first embodiment is below the level of detection of quantification; and the at least one peptide is eliminated from the analysis or assigned a default value. Other features optionally incorporated to this embodiment are as described herein e.g., as in the second or third embodiment. A preselected ratio or a below detection limit result can also be represented for a group of target isoforms.

In a fifth embodiment, the quantitative ratio between the peptides belonging to a group in the first embodiment are used to formulate as a linear or nonlinear regression; and the protein isoform ratios are used as the unknown variables. Other features related to this embodiment are as described in e.g., the second, third, or fourth embodiment. In an alternative, the quantitative ratio between the peptides belonging to a group in the first embodiment is formulated as univariable or multivariable. Other features optionally incorporated to this embodiment are as described herein e.g., as in the second, third, or fourth embodiment.

In a sixth embodiment, quantitative constraints are imposed on the protein isoform ratios described herein, e.g., as described in the first, second, third, or fourth embodiment. Alternatively, quantitative constraints are imposed on the protein isoform ratios described herein, e.g., as described in the first, second, third, or fourth embodiment; and the quantitative ratio between the peptides belonging to a group are used to formulate a linear programming or nonlinear programming model; and wherein the protein isoform ratios are used as the unknown variables.

In a seventh embodiment, the methods described herein, such as those described in the first, second, third, fourth, fifth, or sixth embodiment may be performed by an automated system.

In an eighth embodiment, for each group of selected peptides described in the first embodiment, a number representing the difference in the physiochemical properties of the peptides belonging to a group is calculated. Other features optionally incorporated to this embodiment are as described herein e.g., as in the second, third, fourth, fifth, sixth, or seventh embodiment. Alternatively, for each group of selected peptides described in the first embodiment, a number representing the difference in the physiochemical properties of the peptides belonging to a group is calculated; and a weight reflecting this number is used in the calculation. Other features optionally incorporated to this embodiment are as described herein e.g., as in the second, third, fourth, fifth, sixth, or seventh embodiment. In another alternative, for each group of selected peptides in the first embodiment, a number representing the difference in the physiochemical properties of the peptides belonging to a group is calculated; and the groups are ranked based on the calculated number. Other features optionally incorporated to this embodiment are as described herein e.g., as in the second, third, fourth, fifth, sixth, or seventh embodiment. In yet another alternative, for each group of selected peptides in the first embodiment, a number representing the difference in the physiochemical properties of the peptides belonging to a group is calculated; the groups are ranked based on the calculated number; and calculating the ratio of protein isoforms using the ranked groups that are lower or greater than a threshold. Other features optionally incorporated to this embodiment are as described herein e.g., as in the second, third, fourth, fifth, sixth, or seventh embodiment.

In a ninth embodiment, in the methods described herein, such as those described in the first, second, third, fourth, fifth, sixth, seventh, or eighth embodiment, absolute quantities or concentrations of the one or more protein isoforms are determined.

In a tenth embodiment, in the methods described herein, such as those described in the first, second, third, fourth, fifth, sixth, seventh, eighth, or ninth embodiment, the selected peptides are present in multiple forms.

In an eleventh embodiment, in the methods described herein, such as those described in the first, second, third, fourth, fifth, sixth, seventh, eighth, ninth, or tenth embodiment, the selected peptides comprise different sets of post translational modifications. Alternatively, in the methods described herein, such as those described in the first, second, third, fourth, fifth, sixth, seventh, eighth, ninth, or tenth embodiment, the selected peptides comprise different sets of post translational modifications; and quantitative response for at least one of the selected peptides is added to estimate the total quantitative response for the peptide.

In a twelfth embodiment, in the methods described herein, such as those described in the first, second, third, fourth, fifth, sixth, seventh, eighth, ninth, tenth, or eleventh embodiment, the protease is endogenous.

In a thirteenth embodiment, in the methods described herein, such as those described in the first, second, third, fourth, fifth, sixth, seventh, eighth, ninth, tenth, or eleventh embodiment, the protease is selected from a set of available proteases so that specific peptides can be measured so as to calculate ratio between specific isoforms or specific groups.

In a fourteenth embodiment, the methods described herein, such as those described in the first, second, third, fourth, fifth, sixth, seventh, eighth, ninth, tenth, eleventh, twelfth, or thirteenth embodiment, further comprise pre-treating the sample for purification, enrichment or fractionation.

In a fifteenth embodiment, the methods described herein, such as those described in the first, second, third, fourth, fifth, sixth, seventh, eighth, ninth, tenth, eleventh, twelfth, thirteenth, or fourteenth embodiment, further comprise using peptide standards to normalize the individual peptide measurements. In one aspect of this embodiment, the peptide standard comprises measurements reported from literature values.

In a sixteenth embodiment, isotopically labeled peptide standards are used in the first embodiment to normalize the individual peptide measurements. Other features optionally incorporated to this embodiment are as described herein e.g., as in the second, third, fourth, fifth, sixth, seventh, eighth, ninth, tenth, eleventh, twelfth, thirteenth, fourteenth, or fifteenth embodiment. In one aspect of this embodiment, the peptide standard comprises measurements reported from literature values.

In a seventeenth embodiment, the methods described herein, such as those described in the first, second, third, fourth, fifth, sixth, seventh, eighth, ninth, tenth, eleventh, twelfth, thirteenth, fourteenth, fifteenth, or sixteenth embodiment, the selected peptide groups are obtained by an in-silico digestion of the protein isoforms.

In an eighteenth embodiment, the methods described herein, such as those described in the first, second, third, fourth, fifth, sixth, seventh, eighth, ninth, tenth, eleventh, twelfth, thirteenth, fourteenth, fifteenth, sixteenth, or seventeenth embodiment, the protein isoform sequence is generated in-silico using empirical evidence, known protein databases and biological and algorithmic model of multiple mutations, and rearrangements and splicing events using genomics databases.

In a nineteenth embodiment, the methods described herein, such as those described in the first, second, third, fourth, fifth, sixth, seventh, eighth, ninth, tenth, eleventh, twelfth, thirteenth, fourteenth, fifteenth, sixteenth, seventeenth, or eighteenth embodiment, denovo sequencing is used to sequence peptides which can then be added to previous generated peptide groups based on their sequence homology.

In the methods described herein, peptides can be quantified by methods known to one of skill in the art. For example, the described peptides can be quantified using a variety of methods that include, but are not limited to, chromatography based methods (e.g., Liquid Chromatography followed by Mass Spectrometry (LCMS), Liquid Chromatography followed by Tandem Mass Spectrometry (LCMS/MS), Liquid Chromatography followed by SRM or MRM Mass Spectrometry (LC-SRM or LC-MRM), and the like); mass spectrometry, UV based methods, and fluorescence detection techniques. quantitative method.

In one embodiment, mass spectrometry is used to quantify the peptides in the method described herein. For example, all the peptide species in the sample are ionized, and then each can be quantitatively measured using its intact mass to charge (m/z) of one or more charge states in which that peptide is ionized, or the mass of charge of its fragments (after applying certain energy to fragment the peptide into characteristic m/z values). The total ions are counted or the flux of ions is measured by an automated system, and is then used to calculate the peptide abundance.

In a twentieth embodiment, in the methods described herein, a number related to the digestion efficiencies of the various protein isoforms is used as a weight in the calculations.

In a twenty-first embodiment, in the methods described herein, multiple analyses or experiments are performed to measure peptide ratios of different or same peptide groups, and the results are combined during calculations.

Other methods with the scope of the present disclosure are provided in the Exemplification section below.

### Exemplification

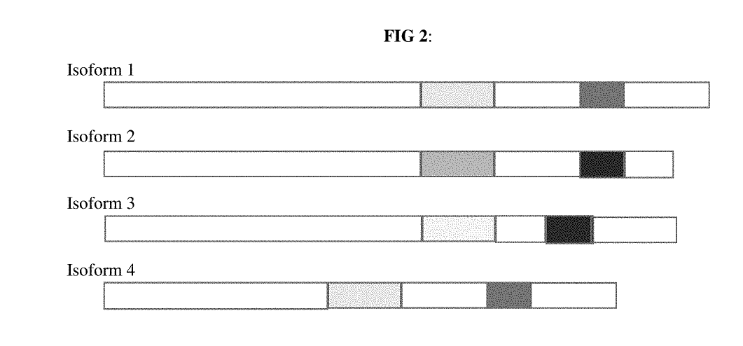

An exemplary method of determining the ratio of one or more isoforms of a protein using the method described herein is shown in FIG. 2. A sample having a protein containing 4 isoforms is provided. The boxes represent protein sequence with some shared peptides. There are two peptide groups depicted by dark and light grey boxes. The dark boxes represent one peptide sequence that is common to isoform 2 and 3, but that is slightly different from the peptide that is common to isoform 1 and 4 (both of which have the same peptide sequence). The light grey boxes represent a completely different peptide sequence that is common to isoform 1 and 4, but that is slightly different in isoform 2 and also slightly different in isoform 3. The quantitative response for each of these peptide sequences can then be used to mathematically model the relative ratios of the 4 protein isoforms according to the following:

Response(p_{Dark1})/Response(p_{Dark2})=(x₂+x₃)/(x₁+x₄)

Response(p_{Light1})/Response(p_{Light2})=(x₁+x₄)/x₂

Response(p_{Light1})/Response(p_{Light3})=(x₁+x₄)/x₃.

### Example 1

Apolipoprotein E is known to be present in three isoforms: ApoE2, ApoE3 and ApoE4. Two samples containing all three isoforms are digested with GluC and processed using standard LCMS based SOP. Digested samples are desalted with C18 solid phase extraction media. High Resolution LC-MS/MS analysis is carried out on an LTQ-Orbitrap XL mass spectrometer, and peptides are identified and quantified using their area under the peak in MS1 (intact precursor). Using GluC, the following peptides are expected to be formed:

Group 1: DVCGRLVQYRGE (E2 and E3) and DVRGRLVQYRGE (E4)

Peptide Signal Response Measured:

| Peptide | Sample 1 | Sample 2 |
|---|---|---|
| DVCGRLVQYRGE | 6e6 | 1e6 |
| DVRGRLVQYRGE | 2e6 | 5e6 |

In a parallel experiment, samples were also digested with Chymotrypsin, and for that we expect the following peptides to be formed:

Group 2: QKC (E2) and QKR (E3 and E4)

Peptide Signal Response Measured:

| Peptide | Sample 1 | Sample 2 |
|---|---|---|
| QKC | 1e5 | 9e4 |
| QKR | 1e5 | 9e5 |

Let the ratio of the three protein isoforms E2, E3 and E4 be 1, x and y respectively (where x and y are unknown variables). Using the ratio of peptide signal from Group 1, we can formulate the following linear regression equations:

For sample 1: (1+x)/y=3 and 1/(x+y)=1

For sample 2: (1+x)/y=0.2 and 1/(x+y)=0.1

Solving these two, gives: Sample 1: x=0.5, y=0.5 and Sample 2: x=0.83, y=9.17

The following isoform ratios are inferred:

| Isoform ratio | Sample 1 | Sample 2 |
|---|---|---|
| E3 to E2 | 0.5 | 0.83 |
| E4 to E2 | 0.5 | 9.17 |

1. A method for determining the ratio of one or more isoforms of a protein in a sample, the method comprising:
digesting the protein with at least one protease to produce a plurality of peptides;
quantifying one of more of the peptides produced;
selecting, from the peptides produced, groups of peptides having sequence homology and at least two peptides;
identifying, for each selected group of peptides, which peptides belong to which one or more target isoforms;
determining the quantitative ratio between peptides belonging to a group; and
calculating the ratio of protein isoforms or ratio of groups of protein isoforms, thereby determining the relative ratio of the one or more isoforms.

2. The method of claim 1, wherein at least one peptide group comprises one or more peptides that are unique to particular isoform.

3. The method of claim 1, wherein the quantitative ratio of two peptides from a single group is used to calculate and report the ratio of two protein isoforms.

4. The method of claim 1, wherein the quantitative ratio of two peptides from a single group is used to calculate and report the ratio of two groups of protein isoforms.

5. The method of claim 1, wherein at least one peptide in the selected group is below the level of detection of level of quantitation.

6. The method of claim 1, wherein at least one peptide in the selected group is below the level of detection of level of quantitation; and the at least one peptide is either eliminated from the analysis or assigned a default value.

7. The method of claim 1, wherein the quantitative ratio between the peptides belonging to a group are used to formulate a linear or nonlinear regression; and wherein the protein isoform ratios are used as the unknown variables.

8. The method of claim 1, comprising quantitative constraints on the protein isoform ratios.

9. The method of claim 1, comprising quantitative constraints on the protein isoform ratios, wherein the quantitative ratio between the peptides belonging to a group are used to formulate a linear programming or nonlinear programming model; and wherein the protein isoform ratios are used as the unknown variables.

10. The method of claim 1, wherein the method is performed by an automated system.

11. The method of claim 1, wherein for each group of selected peptides, a number representing the difference in the physiochemical properties of the peptides belonging to a group is calculated.

12. The method of claim 1, wherein for each group of selected peptides, a number representing the difference in the physiochemical properties of the peptides belonging to a group is calculated; and a weight reflecting this number is used in the calculation.

13. The method of claim 1, wherein for each group of selected peptides, a number representing the difference in the physiochemical properties of the peptides belonging to a group is calculated; and the groups are ranked based on the calculated number.

14. The method of claim 1, wherein for each group of selected peptides, a number representing the difference in the physiochemical properties of the peptides belonging to a group is calculated; the groups are ranked based on the calculated number; and calculating the ratio of protein isoforms using the ranked groups that are lower or greater than a threshold.

15. The method of claim 1, wherein absolute quantities or concentrations of the one or more protein isoforms are determined.

16. The method of claim 1, wherein the selected peptides are present in multiple forms.

17. The method of claim 1, wherein the selected peptides are identified or quantified with different sets of post translational modifications.

18. The method of claim 1, wherein the selected peptides are identified or quantified with different sets of post translational modifications; and quantitative response from at least one of these sets is added to estimate the total quantitative response for the peptide.

19. The method of claim 1, wherein the protease is endogenous and the sample may or may not be subjected to additional external proteases during sample processing.

20. The method of claim 1, wherein the protease is selected from a set of available proteases so that specific peptides can be measured so as to calculate ratio between specific isoforms or specific groups.

21. The method of claim 1, further comprising pre-treating the sample for purification, enrichment or fractionation.

22. The method of claim 1, further comprising using peptide standards to normalize the individual peptide measurements.

23. The method of claim 1, wherein isotopically labeled peptide standards to normalize the individual peptide measurements.

24. The method of claim 1, wherein the peptide standard comprises measurements reported from literature values.

25. The method of claim 1, wherein the selected peptide groups are obtained by an in-silico digestion of the protein isoforms.

26. The method of claim 1, wherein the protein isoform sequence are generated in-silico using empirical evidence, known protein databases and biological and algorithmic model of multiple mutations, and rearrangements and splicing events using genomics databases.

27. The method of claim 1, wherein denovo sequencing is used.

28. The method of claim 1, wherein a number related to the digestion efficiencies of the various protein isoforms is used as a weight in the calculations.

29. The method of claim 1, wherein multiple analyses or experiments are performed to measure peptide ratios of different or same peptide groups, and the results are combined during calculations.
